(19) United States
(12) Patent Application Publication
(10) Pub. No.: US 2019/0091435 A1
(43) Pub. Date: Mar. 28, 2019

(54) INFLATABLE CONDUIT AND HEADGEAR INCLUDING SAME

(71) Applicant: KONINKLIJKE PHILIPS N.V., EINDHOVEN (NL)

(72) Inventors: Daniel Steed, North Huntingdon, PA (US); Farah Asad Khan, Pittsburgh, PA (US)

(21) Appl. No.: 16/142,709

(22) Filed: Sep. 26, 2018

Related U.S. Application Data
(60) Provisional application No. 62/564,303, filed on Sep. 28, 2017.

(51) Int. Cl. A61M 16/08; A61M 16/20; A61M 16/06

(52) U.S. Cl. CPC A61M 16/0875; A61M 16/0683; A61M 16/208; A61M 16/0883

(57) ABSTRACT

A conduit for conveying a flow of a gas therethrough includes an inner wall having a first end and an opposite second end. The inner wall is disposed around, and defines a central passage extending between the first end and the second end. The central passage is structured to convey the flow of the gas therethrough between the first end and the second end. The conduit also includes an outer wall disposed about the inner wall and a sealed compartment defined between the inner wall and the outer wall, the sealed compartment being structured to house a fixed quantity of a gas therein.

### CROSS-REFERENCE TO RELATED APPLICATIONS

This patent application claims the priority benefit under 35 U.S.C. § 119(e) of U.S. Provisional Application No. 62/564,303, filed on Sep. 28, 2017, the contents of which are herein incorporated by reference.

### BACKGROUND OF THE INVENTION

### 1. Field of the Invention

The present invention pertains to conduits for conveying a flow of a gas therethrough, and more particularly, to conduits for conveying a flow of a gas therethrough which are selectively readily collapsible. The present invention also pertains to headgear for use in securing a patient interface device to a head of a patient and to systems for delivering a flow of treatment gas to the airway of a patient.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

In order to increase the comfort of devices, the CPAP market is asking for headgear formed from fabric instead of plastic and rubber. Fabric adds to the tactile and emotional appeal as well as has technical advantages, such as reduced red marks on the skin and sweat absorption. As the CPAP market moves toward apparel commodity, fabrics are becoming more important. Furthermore, the desire for a lightweight, collapsible solution exists for headgear and conduit to make such items more readily transportable and less cumbersome.

### SUMMARY OF THE INVENTION

As one aspect of the present invention, a conduit for conveying a flow of a gas therethrough is provided. The conduit comprises an inner wall having a first end and an opposite second end. The inner wall is disposed around, and defines a central passage extending between the first end and the second end. The central passage is structured to convey the flow of the gas therethrough between the first end and the second end. The conduit further comprises an outer wall disposed about the inner wall and a sealed compartment defined between the inner wall and the outer wall, the sealed compartment being structured to house a fixed quantity of a gas therein.

The inner wall and the outer wall may be formed from one or more materials which are impermeable to gas.

At least one of the inner wall and the outer wall may be formed from a laminate material comprising a layer of fabric material and a layer of silicone or TPE mechanically bonded to the fabric material.

The sealed compartment may extend completely around the inner wall.

The conduit may further comprise a valve member structured to regulate passage of the gas in or out of the sealed compartment.

The valve member may be disposed between the central passage and the sealed compartment.

The valve member may comprise a valve which permits flow from the central passage to the sealed compartment but prevents flow from the sealed compartment to the central passage. The conduit may further comprise a second valve member disposed between the sealed compartment and an exterior of the outer wall.

The valve member may be disposed between the sealed compartment and an exterior of the outer wall.

As another aspect of the present invention, a headgear for use in securing a patient interface device to the head of a patient is provided. The headgear comprises a conduit as recited herein.

As yet another aspect of the present invention, a system for delivering a flow of treatment gas to the airway of a patient is provided. The system comprises: a patient interface device structured to sealingly engage about an airway of the patient; and a conduit having a first end coupled to the patient interface device and an opposite second end structured to be couple to a pressure generating device. The conduit comprises an inner wall having a first end and an opposite second end. The inner wall is disposed around, and defines a central passage extending between the first end and the second end. The central passage is structured to convey the flow of the gas therethrough between the first end and the second end. The conduit also comprises an outer wall disposed about the inner wall and a sealed compartment defined between the inner wall and the outer wall, the sealed compartment being structured to house a fixed quantity of a gas therein.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a simplified diagram of an airway pressure support system according to an exemplary embodiment which is operated within an environment, such as a bedroom or home of the user of airway pressure support system, shown with a patient interface device thereof disposed on the face of a patient;

FIG. 2 is an elevation view of an example of a conventional delivery conduit which may be used in the system of FIG. 1;

FIG. 3 is an elevation view of a portion of the conventional delivery conduit of FIG. 2 shown with portions removed to show internal details;

FIG. 4 is a side elevation view of a conduit in accordance with an example embodiment of the present invention;

FIG. 5 is a sectional view of the conduit of FIG. 4 taken along line 5-5 of FIG. 4;

FIG. 6 is a sectional view of a portion of a conduit in accordance with an example embodiment of the present invention taken along a line parallel to the flow of gas therethough;

FIGS. 7 and 8 are sectional isometric views of conduits in accordance with example embodiments of the present invention;

FIG. 9 is an isometric view of a headgear in accordance with an example embodiment of the present invention; and

FIG. 10 is a sectional view of a portion of the headgear of FIG. 9 taken along line 10-10 of FIG. 9.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments of the present invention provide for a collapsible conduit and/or frame which can be readily inflated and create structure through which CPAP therapy air may flow. Even during the event of a power loss, the conduit and/or frame will remain inflated, maintain its shape, and allow airflow therethrough.

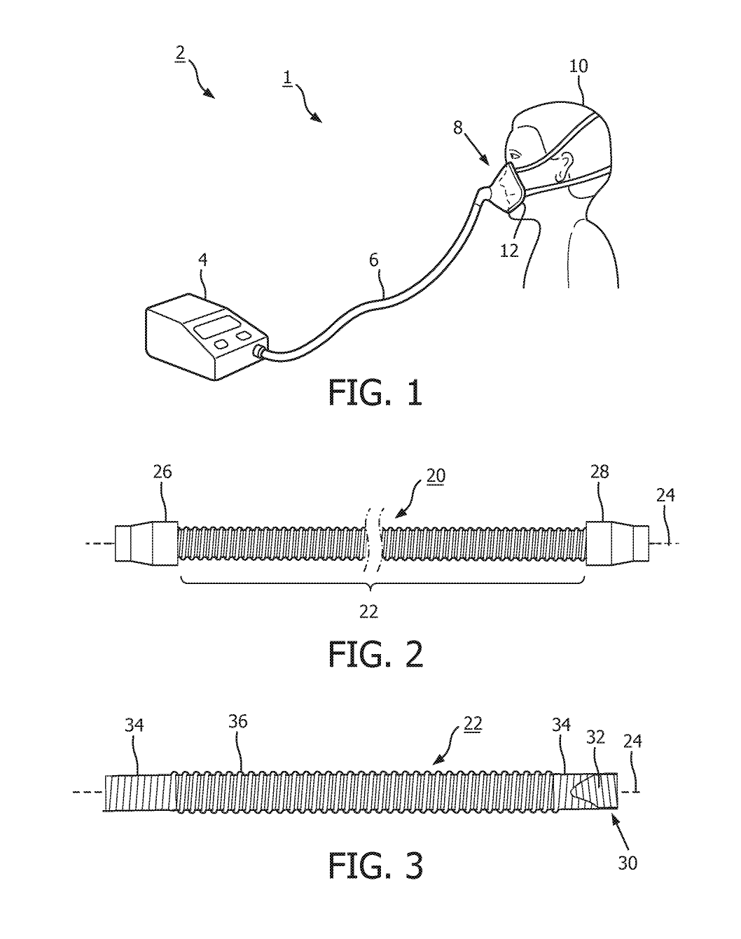

An example airway pressure support system 2 according to one particular, non-limiting exemplary embodiment of the present invention which is operated within an ambient environment 1, such as, without limitation, a bedroom or home of the user of airway pressure support system 2 is shown in FIG. 1. System 2 includes a pressure/flow generator 4, a delivery conduit 6, a patient interface device 8 structured to engage about an airway of the patient, and a headgear 10 for securing patient interface device 8 to the head of a patient (not numbered). Pressure generating device 4 is structured to generate a flow of breathing gas which may be heated and/or humidified. Pressure generating device 4 may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8. Delivery conduit 6 and patient interface device 8 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems. Although described herein in example embodiments wherein a pressurized flow of gas is utilized, it is to be appreciated that embodiments of the invention as described herein could also be readily employed in other generally non-pressurized applications (e.g., without limitation, in high flow therapy applications).

In the exemplary embodiment, patient interface device 8 includes a patient sealing assembly 12, which in the illustrated embodiment is a full face mask. It is to be appreciated, however, that other types of patient sealing assemblies, such as, without limitation, a nasal/oral mask, a nasal cushion, or any other arrangements wherein rainout is a potential concern, which facilitate the delivery of the flow of breathing gas to the airway of a patient may be substituted for patient sealing assembly 12 while remaining within the scope of the present invention. It is also to be appreciated that headgear 10 is provided solely for exemplary purposes and that any suitable headgear arrangement may be employed without varying from the scope of the present invention.

An example of a conventional conduit 20 which is commonly used in system 2 is shown in FIGS. 2 and 3. Referring first to FIG. 2, conduit 20 generally includes a tubular body portion 22 which is disposed about a central longitudinal axis 24. Coupling members 26, 28 may be provided at respective ends of body portion 22 for use in coupling body portion 22 to pressure generating device 4 and patient interface device 8.

Referring now to FIG. 3, body portion 22 generally includes a wall portion 30 which is of tubular shape and thus defines a passage 32 therein which is structured to communicate the flow of breathing gas from pressure generating device 4 to patient interface device 8. Wall portion 30 is generally formed from a thin, flexible strip 34 of material having an outer surface 34A and an opposite inner surface 34B which is disposed helically about central longitudinal axis 24 such that adjacent helical convolutions of strip 34 overlap a predetermined distance (d), and seal against each other (i.e., a portion of outer surface 34A of one convolute seals against inner surface 34B of the adjacent convolute), thus forming a continuous, generally flexible tube. In order to avoid undesirable crimping or crushing of such tube, body portion 22 further includes a support member 36 formed from a stiffer, more rigid material (typically metal or rigid plastic) than strip 34. Support member 36 is disposed in a helical manner about central longitudinal axis 24 along outer surface 34A of strip 34.

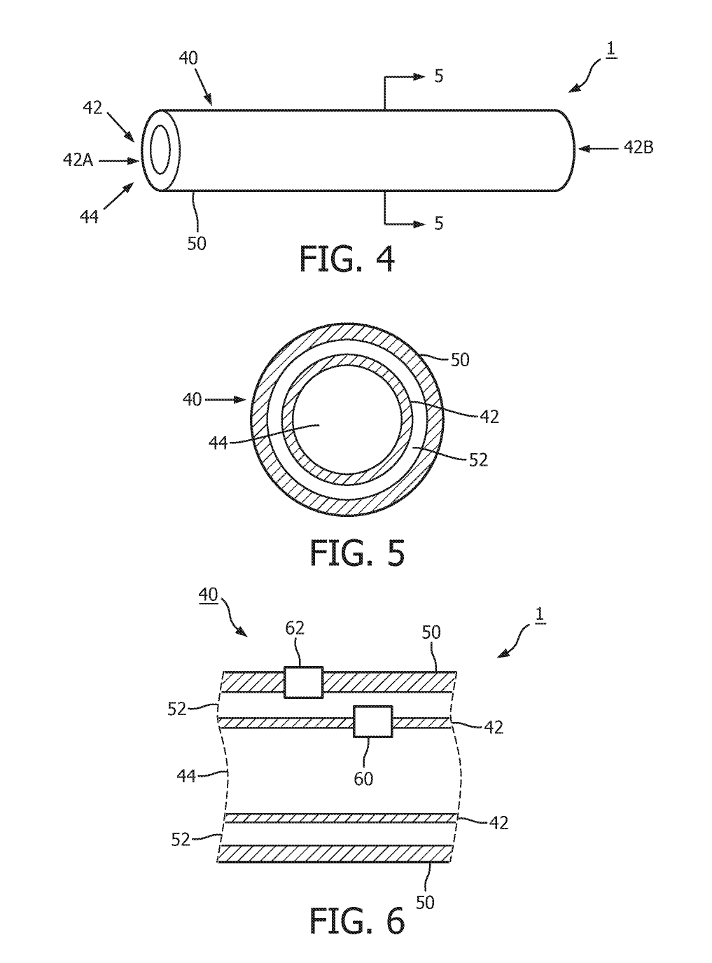

An example embodiment of a conduit 40 for conveying a flow of gas therethrough in accordance with the present invention will now be described in conjunction with FIGS. 4 and 5. Conduit 40 includes an inner wall 42 having a first end 42A and an opposite second 42B. Inner wall 42 is disposed around, and defines a central passage 44 which extends between first end 42A and second end 42B. Central passage 44 is structured to convey the flow of the gas between first end 42A and second end 42B. Conduit 40 further includes an outer wall 50 disposed about inner wall 42 and coupled to inner wall 42 at or about first end 42A and at or about second end 42B such that a sealed compartment 52 is defined between inner wall 42 and outer wall 50 and between first end 42A and second end 42B. As used herein, the phrase “sealed compartment” shall refer to a space which is isolated such that air or any other gas cannot enter or exit such space, hence, a sealed compartment can be said to house a fixed quantity of a gas therein.

In order to contain gas within central passage 44 and sealed compartment 52, each of inner wall 42 and outer wall 50 are formed from one or more materials which are impermeable to gas. Examples of such materials include, but are not limited to: Thermoplastic elastomer (TPE), Silicone, and polyvinyl chloride (PVC)—although PVC would likely not be used for inner wall 42 as it is unlikely to pass biocompatibility testing for airway breathing. Combinations of materials may also be employed (e.g., each layer could be a different material). In example embodiments of the present invention, one or both of inner wall 42 and outer wall 50 haven been formed from laminate materials having a layer of fabric material and a layer of silicone or similar material (e.g., without limitation, TPE) mechanically bonded to the fabric material. The fabric layer of such materials provides for desirable appearances and textures while also providing a framework for the generally thin layer of silicone which provides impermeability to the material.

Referring now to FIG. 6, in order to allow for selective inflation or deflation of sealed compartment 52, conduit 40 may further include one or more valve members, disposed between sealed compartment 52 and central passage 44 and/or the outer surface of outer wall 50 (i.e., between sealed compartment 52 and the ambient environment. For example, inner wall 42 may include a valve member 60 (shown schematically, e.g., a flapper valve or other suitable arrangement) which allows air flow from central passage 44 to sealed compartment 52 but prevents flow from sealed compartment 52 to central passage 44 may be utilized. Such arrangement utilizes the flow of gas through central passage to inflate sealed compartment 52. In such embodiment it is desirable for outer wall 50 to also include a valve 62 (shown schematically, e.g., without limitation, a manually activated valve, an opening having a removable cap, etc.) disposed between sealed compartment 52 and ambient environment 1 to allow for deflation of sealed compartment 52. Alternatively, valve 60 may be structured to allow for selective passage of air (e.g., via selective activation from a user) from sealed compartment 52 to central passage 44, in order to allow for deflation of sealed compartment 52.

As another example, conduit 40 may include only valve member 62 disposed in outer wall 50 which regulates airflow between sealed compartment 52 and the ambient environment 1, providing for the selective inflation or deflation of sealed compartment 52. In such embodiment, inflation of sealed compartment may be accomplished via manual inflation by a user blowing air (e.g., via a valve similar to that of a beach ball or other suitable mechanism) or via a pressure generating device (e.g., via an adaptor coupleable between a pressure generating device such as device 4 of FIG. 1 and valve 62).

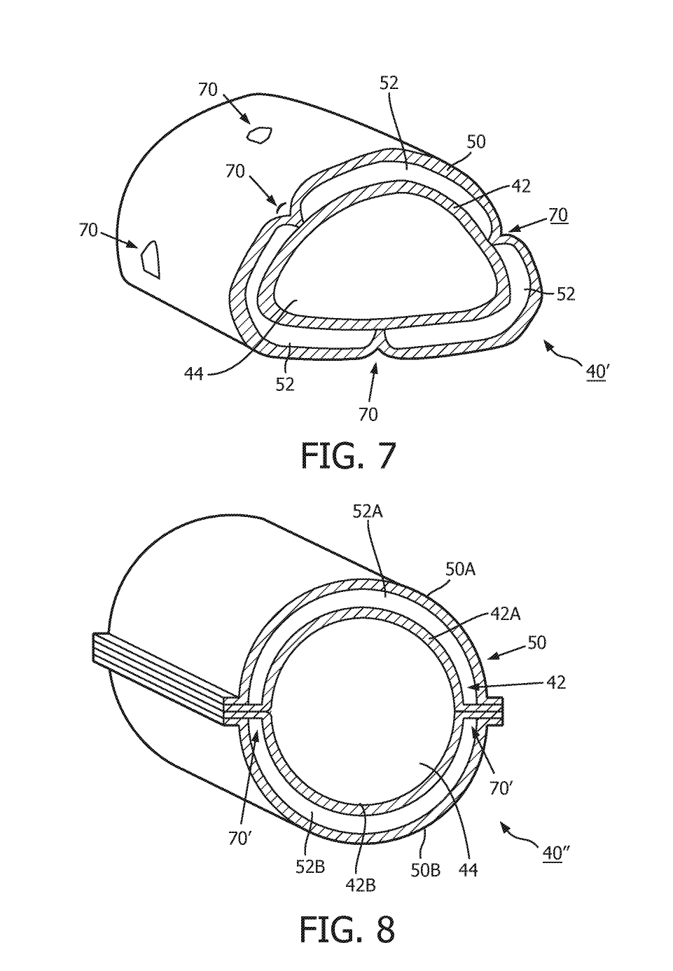

FIG. 7 shows an example of another conduit 40′ in accordance with an example embodiment of the present invention. Conduit 40′ is of a similar arrangement as conduit 40 except inner wall 42 and outer wall 50 are coupled together at several locations 70 to keep inner wall 42 and outer wall 50 in a generally fixed relationship. Such coupling of inner wall 42 and outer wall 50 may be accomplished via any suitable process, e.g., without limitation, via welding, glue, etc.

FIG. 8 shows an example of yet another conduit 40″ in accordance with an example embodiment of the present invention. Conduit 40″ is of a similar arrangement as conduit 40 except inner wall 42 and outer wall 50 are coupled together on opposite sides of central passage 44 along generally linear regions 70′ which extend along conduit 40″. In the example shown in FIG. 8, inner wall 42 includes a first layer of material 42A and a second layer of material 42B which are coupled together at or about linear regions 70′. Similarly, outer wall 50 includes a first layer of material 50A and second layer of material 50B which are coupled together (via first and second layers 42A and 42B in the example of FIG. 8) at or about linear regions 70′. Such couplings may be accomplished via any suitable process, e.g., without limitation, via welding, glue, etc. As a result of such arrangement, two sealed compartments, an upper sealed compartment 52A and a lower sealed compartment 52B are formed by such arrangement with upper sealed compartment 52A being bounded/defined by first layers 42A and 50A and with lower sealed compartment 52B being bounded/defined by second layers 42B and 50B.

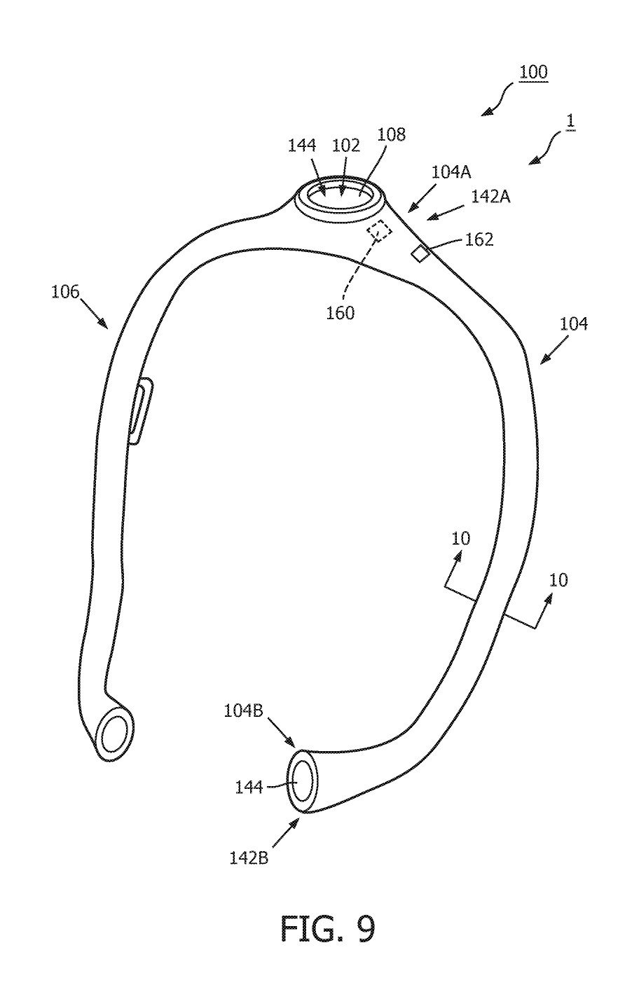

Having thus described several embodiments of conduits according to example of the present invention, an example headgear 100 which can be formed one or more of such conduits is shown in FIG. 9. Headgear 100 includes a top portion 102 and first and second side arms 104 and 106 extending generally downward therefrom. Top portion 102 and side arms 104 and 106 are positioned and arranged to so as to be disposed at the top and respective sides of a patient's head when headgear 100 is disposed on the head of a patient. Top portion 102 includes an inlet 108 defined therein which is structured be coupled to a conduit providing a flow of gas from a pressure generating device. First arm 104 extends from a first end 104A at or about top portion 102 to an opposite second end 104B disposed distant from top portion 102. Second end 104B is positioned and structured to be coupled to a patient interface device (e.g., without limitation, a nasal cushion, or any other suitable interface device) which is adapted to sealing engage about one or more of a patient's oral or nasal orifices.

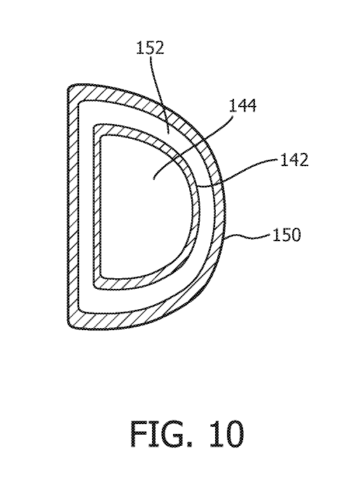

Referring now to the sectional view of FIG. 10 in addition to FIG. 9, first arm 104 is formed generally like conduit 40 previously described in conjunction with FIGS. 4 and 5. Accordingly, first arm 104 includes an inner wall 142 having a first end 142A disposed at first end 104A of first arm 104 and an opposite second 142B disposed at second end 104B of first arm. Inner wall 42 is disposed around, and defines a central passage 144 which extends between inlet 108 in top portion 102 and second end 104B of first arm 104. Central passage 144 is structured to convey the flow of the gas between inlet 108 and second end 104B. First arm 104 further includes an outer wall 150 disposed about inner wall 142 and coupled to inner wall 142 at or about first end 142A and at or about second end 142B such that a sealed compartment 152 is defined between inner wall 142 and outer wall 150 and generally between top portion 102 and second end 104B of first arm 104.

In order to contain gas within central passage 144 and sealed compartment 152, each of inner wall 142 and outer wall 150 are formed from one or more materials which are impermeable to gas, such as those previously discussed herein. In example embodiments of the present invention, one or both of inner wall 142 and outer wall 150 haven been formed from laminate materials having a layer of fabric material and a layer of silicone or similar material (e.g., without limitation, TPE) mechanically bonded to the fabric material. As previously discussed, the fabric layer of such materials provides for desirable appearances and textures while also providing a framework for the generally thin layer of silicone which provides impermeability to the material. Second arm 106 is of similar arrangement as first 104 and thus the arrangement of such is not further described in detail herein. From the foregoing it is thus to be appreciated that head gear 100 is formed essentially as two conduits similar to those previously described herein with each positioned and structured to provide a flow of a gas from inlet 108 of top portion 102 to a distant end of each side arm 104 and 106. It is to be appreciated that sealed compartments 152 of each sidearm 104 and 106 may be segregated at or bout top portion 102 or may be connected so as to form one continuous sealed compartment 152 which extends generally between the distant ends of side arms 104 and 106, with top portion 102 disposed adjacent a mid-portion thereof.

In order to allow for selective inflation or deflation of each, or a single sealed compartment 152, headgear 100 may further include one or more valve members, disposed between sealed compartment 152 and central passage 144 and/or the outer surface of outer wall 150 (i.e., between sealed compartment 152 and the ambient environment. For example, inner wall 142 may include a valve member 160 (shown schematically in dashed line, e.g., a flapper valve or other suitable arrangement) which allows air flow from central passage 144 into sealed compartment 152 but prevents flow from sealed compartment 152 to central passage 144 may be utilized. Such arrangement utilizes the flow of gas through central passage 144 to inflate sealed compartment 152. In such embodiment it is desirable for outer wall 150 to also include a valve 162 (shown schematically, e.g., without limitation, a manually activated valve, an opening having a removable cap, etc.) disposed between sealed compartment 152 and ambient environment 1 to allow for deflation of sealed compartment 152. Alternatively, valve 160 may be structured to allow for selective passage of air (e.g., via selective activation from a user) from sealed compartment 152 to central passage 144, in order to allow for deflation of sealed compartment 152.

As another example, headgear 100 may include only valve member 162 disposed in outer wall 150 which regulates airflow between sealed compartment 152 and the ambient environment 1, providing for the selective inflation or deflation of sealed compartment 152. In such embodiment, inflation of sealed compartment 152 may be accomplished via manual inflation by a user blowing air (e.g., via a valve similar to that of a beach ball or other suitable mechanism) or via a pressure generating device (e.g., via an adaptor coupleable between a pressure generating device such as device 4 of FIG. 1 and valve 62).

From the foregoing, it is to be appreciated that embodiments of the present invention provide for structures for use in conveying a flow of gas therethrough which have selectively rigid sidewalls. When the sealed compartments defined in such sidewalls are inflated, the sidewalls are generally rigid and resistance to collapse. When the sealed compartments defined therein are deflated, the sidewalls and overall structure are readily collapsible and compacted for travel and/or storage purposes.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. It is also to be appreciated that the overall and/or cross sectional shapes of structures described herein are provided for exemplary purposes only and that such shapes may be varied without varying from the scope of the present invention. [52] In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A conduit for conveying a flow of a gas therethrough, the conduit comprising:
an inner wall having a first end and an opposite second end, the inner wall disposed around, and defining a central passage extending between the first end and the second end, the central passage is structured to convey the flow of the gas therethrough between the first end and the second end;
an outer wall disposed about the inner wall; and
a sealed compartment defined between the inner wall and the outer wall, the sealed compartment being structured to house a fixed quantity of a gas therein.

2. The conduit of claim 1, wherein the inner wall and the outer wall are formed from one or more materials which are impermeable to gas.

3. The conduit of claim 2, wherein at least one of the inner wall and the outer wall are formed from a laminate material comprising a layer of fabric material and a layer of silicone or TPE mechanically bonded to the fabric material.

4. The conduit of claim 1, wherein the sealed compartment extends completely around the inner wall.

5. The conduit of claim 1, further comprising a valve member structured to regulate passage of the gas in or out of the sealed compartment.

6. The conduit of claim 5, where the valve member is disposed between the central passage and the sealed compartment.

7. The conduit of claim 6, wherein the valve member comprises a valve which permits flow from the central passage to the sealed compartment but prevents flow from the sealed compartment to the central passage.

8. The conduit of claim 7, further comprising a second valve member disposed between the sealed compartment and an exterior of the outer wall.

9. The conduit of claim 5, where the valve member is disposed between the sealed compartment and an exterior of the outer wall.

10. A headgear for use in securing a patient interface device to the head of a patient, the headgear comprising a conduit as recited in claim 1.

11. A system for delivering a flow of treatment gas to the airway of a patient, the system comprising:
a patient interface device structured to sealingly engage about an airway of the patient; and
a conduit having a first end coupled to the patient interface device and an opposite second end structured to be couple to a pressure generating device, the conduit comprising:
an inner wall having a first end and an opposite second end, the inner wall disposed around, and defining a central passage extending between the first end and the second end, the central passage is structured to convey the flow of the gas therethrough between the first end and the second end;
an outer wall disposed about the inner wall; and
a sealed compartment defined between the inner wall and the outer wall, the sealed compartment being structured to house a fixed quantity of a gas therein.

12. A headgear for use in securing a patient interface device to a head of a patient, the headgear comprising:
a top portion having an inlet defined therein which is structured be coupled to a conduit providing a flow of gas from a pressure generating device;
a first side arm extending generally downward from a first end at or about the top portion to an opposite second end disposed distant from the top portion, the second end is positioned and structured to be coupled to the patient interface device, the first arm comprising:
an inner wall having a first end disposed at the first end of the first arm and an opposite second disposed at the second end of the first arm, the Inner wall being disposed around, and defining a central passage which extends between the inlet defined in the top portion and the second end of the first side arm; and
an outer wall disposed about the inner wall and coupled to the inner wall at or about the first end and at or about the second end such that a sealed compartment is defined between the inner wall and the outer wall and generally between the top portion and the second end of the first side arm,
wherein the top portion is positioned so as to be disposed at or about a top portion of the head of the patient when the headgear is disposed on the head of the patient,
wherein the first side arm is positioned so as to be disposed on a side of the head of the patient when headgear is disposed on the head of the patient, and
wherein the central passage of the first side arm is structured to convey a flow of a gas between the inlet of the top portion to a patient interface device coupled to the second end of the first arm.

13. The headgear of claim 12, wherein the inner wall of the first side arm and the outer wall of the first side arm are formed from one or more materials which are impermeable to gas.

14. The headgear of claim 13, wherein at least one of the inner wall and the outer wall of the first side arm are formed from a laminate material comprising a layer of fabric material and a layer of silicone or TPE mechanically bonded to the fabric material.
